(19) United States
(12) Patent Application Publication
(10) Pub. No.: US 2004/0180095 A1
(43) Pub. Date: Sep. 16, 2004

(54) Treatment of inoperable turmors by stereotactic injection of microspheres

(76) Inventors: Philippe Menei, Angers (FR); Jean-Pierre Benoit, Avrille (FR)

Correspondence Address: BURNS DOANE SWECKER & MATHIS L L P, POST OFFICE BOX 1404, ALEXANDRIA, VA, 22313-1404, US

(21) Appl. No.: 10/451,216

(22) PCT Filed: Dec. 21, 2001
(86) PCT No.: PCT/IB01/02810

(30) Foreign Application Priority Data
Dec. 22, 2000 (US) 60257167

(51) Int. Cl. A61K 9/16; A61K 9/50

(52) U.S. Cl. 424/490

(57) ABSTRACT

Use of biodegradable microspheres releasing an anticancer agent for the manufacture of a medicament for the use in the treatment in humans of inoperable tumors, wherein biodegradable microspheres are administered by stereotactic injection directly into the tumor, into the peritumoral area or at the same time into the tumor and the peritumoral area.

The present patent application relates to the treatment in human of inoperable tumors, especially brain tumors such as glioblastomas, tumors of the otorhinolaryngologic sphere, rectal tumors, osseous, hepatic or brain metastasis, or non malignant cystic tumors like craniopharyngiomas.

Glioblastoma is among the group of rare diseases listed by the National Organization for Rare Disorders.

Malignant glial tumors are primitive tumors of the central nervous system representing, depending on the series, 13 to 22% of intracranial tumors. Histologically, two types of malignant glial tumor are in fact distinguished, anaplasic astrocytomas and glioblastomas, the latter representing the least differentiated form of these tumors.

There is currently no effective treatment against malignant glial tumors. Patients suffering from glioblastoma do not survive for more than one year, even when surgery is combined with chemotherapy and radiotherapy.

The treatment of malignant glial tumors is mainly limited by three phenomena.

The first is the existence of a blood-brain barrier (BBB) which isolates the central nervous system from the rest of the body. This BBB allows only small liposoluble molecules to pass through. The other molecules must be administered in very high doses in order to reach the central nervous system, this administration being at the cost of major systemic side effects.

The second factor limiting the efficacy of the treatment of glial tumors is the infiltrating nature of these tumors. Since the brain is a highly functional organ, it is impossible to perform extensive surgery on it within the carcinological meaning of the term. The most complete excision possible will only be a macroscopically complete excision, leaving a large number of infiltrated tumor cells in the walls of the excision cavity. Many authors have moreover shown that 90% of the malignant glial tumors operated on and treated with radiotherapy showed a recurrence within two centimeters of the original tumor locus.

The final factor limiting the efficacy of the treatment of glial tumors is the low therapeutic index. The tumor cells hide themselves in some way behind normal tissue which is extremely fragile and sensitive to attack, brought about, for example, by radiotherapy or by certain anticancer agents. Thus, it is difficult to destroy the tumor cells without destroying the normal nerve cells.

The progress made in the treatment of glial tumors is insufficient (Kornblith P. L., Walker M., Chemotherapy for malignant gliomas. J. Neurosurg., 68: 1-17, 1988; Shapiro W. R., Green S. B., Burger P. C., Selker R. G., VanGilder J. C., Robertson J., Mahaley S. M., A randomized comparison of intra-arterial versus intravenous BCNU with or without intravenous 5-fluorouracil, for newly diagnosed patients with malignant glioma, J. Neurosurg., 76: 772-781, 1992).

Currently, the standard treatment of glioblastomas following a surgical excision is based on an external radiotherapy. It does not allow a survival time of longer than one year to be achieved. The combination of a radiotherapy with a chemotherapy with 1-(2-chloroethyl)-3-cyclohexyl-1-nitrosourea (BCNU) is effective only on anaplasic astrocytomas. It makes only a modest contribution since it only raises the percentage of survivors at eighteen months, without modifying the survival time.

Moreover, immunotherapy has never established itself in this area and gene therapy has yet to prove itself.

Several techniques aimed at increasing the local concentration of anticancer agents have been investigated, such as osmotic rupture of the blood-brain barrier, injection into the cerebrospinal fluid, intracarotid infusion and intratumoral administration using subcutaneous reservoirs (Tamargo R. J. and Brem H., Drug delivery to the central nervous system, Neurosurgery Quarterly, 2: 259-279, 1992). None of these techniques has been able to increase the survival time of patients and some of them have proven to be highly toxic.

In the last few years, pharmaceutical pharmacy research has enabled the development of implantable polymer systems for protecting active substances from degradation, and for allowing their controlled local release over a given period, while at the same time reducing the systemic side effects. The advantages of these implantable polymer systems have recently inspired several teams to study their application in pathologies of the central nervous system (Langer R., Polymer implants for drug delivery in the brain, J. Controlled Release, 16: 53-60, 1991). In particular, such systems implanted into the tumor excision wall of malignant gliomas delay the recurrence of the tumor and prolong the survival of the patients. Isolated malignant cells persist around the operating cavity, these cells being responsible for 90% of recurrences, which arise within two centimeters of the operating locus. In this region, the nerve tissue is functional and the blood-brain barrier is still intact, which limits the action of conventional chemotherapy and radiotherapy.

Various implantable polymer systems which release active molecules have been developed and tested on animals.

A system of biodegradable disks composed of PCPP-SA (polyl[1,3-bis(carboxyphenoxy)propane-co-sebacic acid]) which release BCNU (Gliadel®) has been developed, despite modest results in clinical studies (Brem H., Polymers to treat brain tumors, Biomaterials 11: 699-701, 1990; Brem H., Mahaley M. S., Vick N. A., Black K. L., Schold S. C., Eller T. W., Cozzens J. W., Kenealy J. N. Interstitial chemotherapy with drug polymer implants for the treatment of recurrent gliomas, J. Neurosurg. 74: 441-446, 1991; Brem. H., Walter K. A., Langer R., Polymers as controlled drug delivery devices for the treatment of malignant brain tumors, Eur. J. Pharm. Biopharm., 39 (1): 2-7, 1993, Brem H., Piantadosi S., Burger P. C., Walker M. et al., Placebo-controlled trial of safety and efficacy of intraoperative controlled delivery by biodegradable polymers of chemotherapy for recurrent glioma, Lancet, 345: 1008-1012, 1995).

Microspheres which release BCNU have been developed, but the results of the animal studies were not particularly encouraging (Torres A. I., Boisdron-Celle M., Benoit J. P., Formulation of BCNU-loaded microspheres: influence of drug stability and solubility on the design of the microencapsulation procedure, J. Microencapsulation, 13: 41-51, 1996; Painbéni T., Venier-Julienne M. C., Benoit J. P., Internal morphology of poly (D, L-lactide-co-glycolide) BCNU-loaded microspheres. Influence on drug stability, Eur. J. Pharm. Biopharm, 1998, 45, 31-39).

Microspheres which release 5-fluorouracile (5-FU) have been developed by the inventors and are disclosed in WO 00/69413. These microspheres are implanted into the operating locus by intratissue injection before radiotherapy, i.e. after resection of the tumor.

By virtue of using these microspheres, the inventors have succeeded, entirely advantageously, in doubling the survival time of patients suffering from a glioblastoma. The reason for this is that the use of the microspheres according to the invention makes it possible to achieve a survival time of at least 90 weeks.

Recently Emerich D. F., Winn S. R., Snodgrass P., LaFreniére D., Agostino M., Wiens T., Xiong H. and Bartus R. T. in Injectable chemotherapeutic microspheres and glioma II: Enhanced survival following implantation into deep inoperable tumors (Pharm. Res., 2000, 17, n°7, 776-781) showed that delivering chemotherapeutics into inoperable tumors in rat enhances survival.

Now the inventors surprisingly showed that these microspheres give also good results in human without resection of the tumor in case of inoperable tumors, especially brain tumors such as glioblastomas, tumors of the otorhinolaryngologic sphere, rectal tumors, osseous, hepatic or brain metastasis, or non malignant cystic tumors like craniopharyngiomas.

Consequently, the present invention relates to the use of biodegradable microspheres releasing an anticancer agent for the manufacture of a medicament for the use in the treatement in humans of inoperable tumors, wherein biodegradable microspheres are administered by stereotactic injections, directly into the tumor or into the peritumoral area or at the same time into the tumor and the peritumoral area.

According to the present invention, inoperable tumors are deep tumors or tumors which are located into functional zones, like functional zones of brain.

As an example of inoperable tumors, the following may be cited: brain tumors such as glioblastomas, tumors of the otorhinolaryngologic sphere, rectal tumors, osseous, hepatic or brain metastasis, or non malignant cystic tumors like craniopharyngiomas.

In a preferred embodiment according to the invention, the tumor is a brain tumor.

In a more preferred embodiment, the brain tumor is one of glioblastomas, metastasis and non malignant cystic tumors like craniopharyngiomas.

In another embodiment of the invention, the microspheres releasing anticancer agent are disclosed in WO 00/69413.

These biodegradable microspheres are coated with a polymer which delays the release of the anticancer agent and maintains, in the parenchymal space, a therapeutically effective concentration for a period of time of at least three weeks, preferably of at least four weeks.

The polymer is chosen from ethylcellulose, polystyrene, poly(ε-caprolactone), poly(d,1-lactic acid) and poly(d,1-lactic acid-co-glycolic acid). The polymer is preferably poly(d,1-lactic acid-co-glycolic acid), or PLAGA, which is a biodegradable polymer permitted in the formulation of sustained-release galenic preparations (unlike PCPP-SA, which is not approved for large-scale clinical use).

The poly(d,1-lactic acid-co-glycolic acid) is preferably 50:50PLAGA (i.e. containing an equal amount of lactic acid and of glycolic acid), for example Resomer® RG 506 supplied by BI Chimie, France, which has a weight-average molecular mass equal to 72 000, a polydispersity index equal to 1.8 and an inherent viscosity of 0.80 dl/g (0.1% solution of polymer in chloroform at 25° C.).

PLAGA is a hydrophobic copolymer, the degradation of which, caused by a hydrolysis reaction, gives rise to two normal biological substrates, lactic acid and glycolic acid, which are metabolized at the end of aerobic glycolysis to CO₂ and H₂O. Studies, which are already long-established, have shown that the respiratory pathway is the main pathway of elimination of these two substrates. The rate of biodegradation of PLAGA depends on the respective proportions of lactic acid and glycolic acid. PLAGA is completely biocompatible and causes a moderate foreign body reaction (Visscher G E, R L Robinson, H V Mauding, Fong J W, Pearson J E, Argentieri G J, Biodegradation of and tissue reaction to 50:50poly(DL-lactide-co-glycolide) microcapsules, J. Biomed. Mat. Res. 19: 345-365, 1985). PLAGA is a constituent element of surgical sutures (Frazza E J, Schmidt E E, A new absorbable suture, J. Biomed. Mater. Res., 5: 43-58, 1971) and of subcutaneously implantable galenic forms (Jalil R, Nixon J R, Biodegradable poly(lactic acid) and poly(lactide-co-glycolide) microcapsules: problems associated with preparative techniques and release properties (Review), J. Microencapsulation, 7: 297-325, 1990). It has been demonstrated that 50:50PLAGA microspheres may be sterilized by γ-irradiation, and that, once implanted by stereotaxy into the brain of a rodent, they are completely biodegraded within two months, causing only moderate reaction of the nonspecific astrocyte and histiocyte type (Menei P, Daniel V, Montero-Menei C, Brouillard M, Pouplard-Barthelaix A, Benoit J P: Biodegradation and brain tissue reaction to poly(DL-lactide-co-glycolide) microspheres, Biomaterials 14: 470-478, 1993; Menei P, Croue A, Daniel V, Pouplard-Barthelaix A, Benoit J P: Fate and biocompatibility of three types of microspheres implanted into the brain, J. Biomed Mat Res, 28, 1079-1085, 1994). The latter result has since been confirmed by Kou J H, Emmett C, Shen P et al., Bioerosion and biocompatibility of poly(d,1-lactic-co-glycolic acid) implants in brain, J Control Release, 43, 123-130, 1997.

The biodegradable microspheres used in the instant invention preferably have a mean diameter of 48±20 μm, preferably 46±7 μm. They contain 15 to 35% by weight of anticancer agent, preferably from 19 to 27% of 5-FU, even more preferably 20% of 5-FU, and 65 to 85% by weight of polymer.

In another more preferred embodiment, the microspheres are prepared by a method consisting in preparing an organic phase in which the anticancer agent and the polymer are dispersed in an organic solvent. The organic phase and an aqueous phase are emulsified, and then the organic solvent is extracted by adding water. Finally, the suspension of microspheres obtained is filtered.

The anticancer agent is dispersed in the organic solvent, with vigorous stirring, before the polymer is added.

Depending on the adaptations made to the method of the prior art, the active principle is ground in a planetary ball mill. The size of the crystals obtained is between 15 and 50 μm. The size of the crystals to be encapsulated and their dispersion are, in fact, essential criteria for controlling the degree of encapsulation and the in vitro release kinetics.

The active principle is then dispersed in an organic solvent, preferably dichloromethane, in a round-bottomed tube, with stirring using a homogenization rod, before the polymer is added.

The homogenization gives a homogeneous suspension, attenuates the differences from one grinding batch to another and reduces the size of the crystals of the active principle.

The organic phase is prepared in a solvent without cosolvent. The absence of cosolvent slows down the precipitation of the polymer during the emulsification phase, such that the particles obtained are less porous.

The active principle dispersion is transferred into a first reactor.

The polymer is added in a proportion by mass of between 8 and 13%, preferably equal to 11%. The organic phase obtained is maintained at room temperature with constant stirring for 2 to 4 hours and then for approximately 15 minutes at a temperature of between 1 and 5° C., preferably equal to 2° C. A longer period of stirring of the organic phase at room temperature ensures total solubilization of the polymer in the solvent.

The aqueous phase is prepared in a second reactor, preferably maintaining it at the same temperature as the organic phase, preferably at 2° C. Reduction of the temperature of the aqueous phase and of the organic phase causes an increase in their viscosity and an increase in the degree of encapsulation. The aqueous phase is, for example, an aqueous 10% PVA solution.

Two jacketed reactors are used and a coolant liquid circulates in series in the two reactors. The temperature of the organic and aqueous phases is advantageously identical, preferably equal to 2° C., when the two phases are mixed together. Good control of the temperature effectively conditions the particle size, the rate of dissolution of the active principle and the extraction speed of the solvent all at once.

The organic phase is transferred from the first reactor into the second. The aqueous phase/organic phase proportion by volume is between 80/3 and 120/3, preferably equal to 100/3.

The emulsion obtained is stirred for at least 3 minutes, preferably for 3 to 6 minutes, even more preferably for 5 minutes. The choice of this period of time is directly correlated with the release kinetics and in particular the “burst” effect over 24-48 hours.

The absence of cosolvent coupled with a sufficient emulsification time allows dissolution of the active principle which is at the surface or poorly coated, such that the release kinetics in the initial phase are better controlled.

Water is added to the emulsion, in an emulsion/water ratio by volume of between ¼ and ½, preferably equal to ⅓, in order to extract the organic solvent. The temperature of the extraction water is between 1 and 5° C., preferably equal to 4° C.

The emulsification and extraction steps are carried out in the same reactor so as to limit the variability from one batch to another and to save time. The temperature of the extraction water is low, so as to limit an excessive dissolution of the active principle.

The suspension of microspheres obtained is mixed for a few minutes and then filtered under an inert atmosphere. Working under an inert atmosphere makes it possible to limit the risks of contamination of the product.

The microspheres, which may be obtained according to the method described above, are advantageously lyophilized.

10 ml of sterile water are added to 2 to 5 g of miicrosphere powder (filtercake). This mixture is frozen at −40° C. and then introduced into the freeze-dryer. The lyophilization lasts 18 hours. At the end of the operation, the secondary drying temperature should be maintained below 10° C.

The microspheres should be stored at +4° C., even when dry.

The microspheres preferably used in the context of the invention contain an anticancer agent which is preferably hydrophilic and/or does not cross the blood-brain barrier. Advantageously, the anticancer agent has no central neurotoxicity. This anticancer agent preferably acts on dividing cells.

The anticancer agent consists of a radiosensitizing anticancer compound or a mixture of anticancer compounds containing at least one radiosensitizing anticancer compound, said anticancer compound(s) being chosen, for example, from 5-fluorouracil (5-FU), platinum agents, such as carboplatin and cisplatin, taxanes, such as docetaxel and paclitaxel, gemcitabine, VP16, mitomycin, idoxuridin, topoisomerase 1 inhibitors, such as irinbtecan, topotecan and camptothecines, nitrosoureas, such as BCNU, ACNU or MCNU, methotrexate, bleomycin, adriamycin, cytoxan and vincristine, immunomodulatory cytokines, such as IL2, IL6, IL12 and IL13, and interferons.

The anticancer agent is preferably 5-fluorouracil (5-FU).

In the context of the present invention, the 50:50 PLAGA microspheres vehiculing 5-FU are particularly preferred. 5-FU is an old and well-known antimitotic agent. It is a hydrophilic molecule which crosses the blood-brain barrier very weakly, and its activity is thus increased by local administration (Bourke R. S., West C. R., Chheda G. et al., Kinetics of entry and distribution of 5-fluorouracil in CSF and brain following intravenous injection in primate, Cancer Res., 33: 1735-1746, 1973; Gerosa M. A., Dougherty D. V., Wison C. B., Rosenblum M. L., Improved treatment of a brain tumor model, Part 2: Sequential therapy with BCNU and 5-fluorouracil, J. Neurosurg., 58: 368, 1983; Kotsilimbas D. G., Karpf R., Meredith S., Scheinberg L. C., Evaluation of parenteral 5-FU on experimental brain tumors, Neurology, 16: 916-918, 1966; Levin V. A., Edwards M. S., Wara W. M., Allen J., Ortega J., Vestnys P., 5-fluorouracil and 1-(2-chloro-ethyl)-3-cyclohexyl-1-nitrosourea (CCNU) followed by hydroxyurea, misonidazole and irradiation for brain stem gliomas: a pilot study of the brain tumor research center and the children cancer group, Neurosurgery, 14: 679-681, 1984; Oda Y., Tokuridi Y., Tsuda E., Handa H., Kieler J., Trial of anticancer pellet in malignant brain tumours, 5-FU and urokinase embedded in silastic. Proceeding of the 6th European Congress of Neurosurgery, Acta neurochirurgica, Suppl. 28: 489-490, 1979; Penn R. D., Kroin J. S., Harris J. E., Chiu K. M., Braun D. P., Chronic intratumoral chemotherapy of a rat tumor with cisplatin and fluorouracil, Appl. Neurophysio., 46: 240-244, 1983; Shapiro W. R., Studies on the chemotherapy of experimental brain tumors: Evaluation of 1-(2-chloroethyl)-3-cyclohexyl-1-nitrosourea, vincristine and 5-fluorouracil, J. Nat. Cancer Institute, 46(2), 359-368, 1971; Shapiro W. R., Green S. B., Burger P. C., Selker R. G., VanGilder J. C., Robertson J. T., Mahaley S. M., A randomized comparison of intra-arterial versus intravenous BCNU with or without intravenous 5-fluorouracil, for newly diagnosed patients with malignant glioma, J. Neurosurg., 76: 772-781, 1992; Soloway A. H., Mark V. H., Dukat E. G. et al., Chemotherapy of brain tumors. I-Transplanted murine ependymoblastomas, Cancer Chemother. Rep., 36: 1-4, 1964).

The activity of 5-FU is also increased by sustained administration.

5-FU is essentially active on tissues which undergo rapid renewal and is exceptionally neurotoxic. 5-FU intervenes in the synthesis of nucleic acids, of which tissues undergoing rapid growth have particular need in order to ensure their proliferation and regeneration. Needless to say, this is not the case for cerebral tissue, in which mitosis is rare in the normal state and occurs only in the glial population. The toxic effects of 5-FU which limit its systemic administration are essentially hematological and gastrointestinal. Although rare neurological side effects of 5-FU have been published, their etiopathogenicity, which is relatively unknown, is probably multifactorial (blockage of the Krebs cycle by a 5-FU catabolite or exacerbation of a preexisting thiamine deficiency) (Aoki N., Reversible leukoencephalopathy caused by 5-fluorouracil derivatives, presenting as akinetic mutism, Surg. Neurol., 25: 279-282, 1986; Moore D. H., Fowler W. C., Crumpler L. S., 5-fluorouracil neurotoxicity, case report, Gynecol. Oncology, 36: 152-154, 1990).

Finally, 5-FU is radio-sensitizing (Koutcher J. A., Alfieri A. A., Thaler H. et al., Radiation enhancement by biochemical modulation and 5-FU, Int. J. Radit. Biol. Phys., 39: 1145-1152, 1997). The superiority of the 5-FU/radiotherapy combination in each of these isolated treatments has been demonstrated since the 1960s on animal models and on tumor cells in vitro (Bagshaw M., A possible role of potentiation in radiation therapy, Amer. J. Roentgenol, 85: 822-833, 1961; Vietti T., Eggerding F., Valeriote F., Combined effect of X-radiation and 5-fluorouracil on survival of transplanted leukemic cells, J. Natl. Inst., 47: 865-870, 1971). This synergism is thought to be due to synchronization of the tumor cell population and to a decrease in the mechanisms of cell repair with 5-FU. The combination of radiotherapy and antipyrimidine (5-FU or BrudR) has already been attempted in man (Goffman T. E., Dachowski L. J., Bobo H et al., Long term follow-up on national cancer institute phase I/II study of glioblastoma multiforme treated with iododeoxyuridine and hyperfractionated irradiation, J. Clinical Oncology, 10: 264-268, 1992). The absence of clear-cut efficacy can also be explained here by the systemic route of administration of the drugs.

When the anticancer agent is 5-FU, the concentration of anticancer agent in the cerebrospinal fluid, which is a reflection of the concentration in the parenchymal space, is between 3 and 20 ng/ml.

In order to limit the neurotoxicity of the anticancer agent contained in the microspheres used in the context of the invention, a neuroprotective compound can advantageously be added to said anticancer agent. This neuroprotective compound is chosen, for example, from peptide growth factors, such as NGF or BDNF.

Advantageously, the microspheres are suspended in a sterile solution, the suspension being administered directly into the tumor or into the peritumoral area or at the same time into the tumor and the peritumoral area.

The sterile solution preferably contains:

between 1 and 1.5%, preferably 1.25% weight/volume, of a viscosity modifier, for example sodium carboxymethyl cellulose,

between 0.5 and 1.5%, preferably 1%, of a 5 surfactant, for example Polysorbate 80®, and

between 3.5 and 4.5%, preferably 4%, of an isotonicity agent, for example mannitol.

The sterile solution has a cinematic viscosity comprised between 1 000 and 2 500 mPa, preferably between 2 000 and 2 500 mPa.

The microspheres are preferably suspended at the time of use, immediately before injection. The suspension preferably contains 3 ml of the sterile solution described above and 700 to 800 mg of biodegradable microspheres.

When the anticancer agent is 5-FU, the total dose of suspension injected corresponds to an amount of 5-FU of between 50 and 200 mg.

The microspheres used in the context of the invention can be prepared by an emulsification-extraction technique, according to a variant of the process described by Boisdron-Celle M., Menei P., Benoit J. P.: Preparation of biodegradable 5-fluoro-uraci1-loaded microspheres, J. Pharm. Pharmacol., 47, 108-114, 1995.

One or more repeated stereotactic injections of microspheres are made either directly into the tumor, or into the peritumoral tissue or at the sames times directly into the tumor and into the peritumoral tissue.

In case of the treatment of brain tumors, the stereotactic procedure is identical to the one used for biopsy of brain tumors; after a premedication, the stereotactic frame is placed under local anesthesia and the coordinates are determined by Magnetic Resonance Imaging (MRI); a cradle is then placed according to said coordinates and after the drill of the skull, the needle is positioned at the level of the tumor and/or at the level of its surrounding area, and the microspheres are injected. The injection may take place in the same time as the biopsy itself.

The tumoral volume being defined with a preoperative MRI, 5 implantation paths are chosen thanks to the stereotactic procedure in order to be distributed at best. According to anatomical conditions, the 5 implantations will be done through a trepan hole or through several drills holes directly into the tumor and inot the peritumoral area. The total volume of the microspheres suspensions will be distributed on these 5 paths (for example, 0.5 ml for 1 path distributed on a height of 1 to 5 cm with a Backlund ELEKTA® needle, at a volume of 100 μl in 5 mn). At the end of the stereotactic procedure, a control is realized with a scanner.

According to the invention, the treatment with microspheres may be followed by a radiotherapy.

The radiotherapy may begin the following day since there is no problem of healing.

A 6-weeks radiotherapy may begin 7 days after the implantation of the microspheres (for example, 60 Gy fractionated into 1.8 Gy 5 days a week). The irradiated volume roughly corresponds to the volume of the tumor evaluated by MRI.

A clinical supervision takes place during the radiotherapy. A clinical check-up is done 24 hours, 10 days, 20 days, 30 days, 3 months, then every 3 months until 1 year after the implantation. A MRI is realized 10 days, 30 days, 3 months and every 3 months until 1 year after the implantation. Blood and CSF samples are taken 10 days and 30 days after the implantation and on the last irradiation day.

The results of a pilot study in man comprising the injection of microspheres of 5-FU followed by a radiotherapy 7 days after the injection, said study including a patient with a cystic tumor, two patients with complete tumors and one patient with a median tumor show a good tolerance, since 5-FU is neither detectable in the CSF nor in the blood of the patients.

Systemic chemotherapy is ineffective, in large part because the concentrations of chemotherapeutic drugs within the tumor and surrounding tissue are not high enough to kill tumor cells. Interstitial chemotherapy using injectable microspheres elevates drug concentrations locally within the tumor for a prolonged period of time.

The ability to easily inject microspheres into the tissue within and surrounding brain tumors increases the likelihood of delivering adequate drug concentrations to tumor cells as they migrate from the primary tumor mass. Moreover, multiple injections of microspheres could be made that intentionally target those regions of higher bulk flow in an attempt to minimize successful tumor cell migration. The ability to sculpt a drug field that encompasses the tumor, the peritumoral region and nearby areas of probable tumor cell migration, maximizes the opportunity to treat both the primary tumor site and its most likely route of infiltration.

These results establish that the microspheres can be easily injected in man into the brain to provide sustained release of the chemotherapeutic drug like 5-FU into a deep inoperable tumor bed, that the injection into the tissue surrounding the tumor and the injection at the same time into the tumor and into the peritumoral tissue may be also effective.

1. Use of biodegradable microspheres releasing an anticancer agent for the manufacture of a medicament for the use in the treatment in humans of inoperable tumors, wherein biodegradable microspheres are administered by stereotactic injection directly into the tumor, into the peritumoral area or at the same time into the tumor and the peritumoral area.

2. Use according to claim 1 wherein the biodegradable microspheres are coated with a polymer which delays the release of the anticancer agent and maintains, in the parenchymal space, a therapeutically effective concentration for a period of time of at least three weeks, preferably of at least four weeks.

3. Use according to claims 1 or 2, wherein the inoperable tumors are deep tumors or tumors which are located into functional zones.

4. Use according to claim 3, wherein inoperable tumors are brain tumors such as glioblastomas, tumors of otorhinolaryngologic sphere, rectal tumors, osseous, hepatic or brain metastasis, or non malignant cystic tumors like craniopharyngiomas.

5. Use according to claim 4, wherein the tumor is a brain tumor.

6. Use according to claim 5, wherein the brain tumor is one of glioblastomas, metastasis and non malignant cystic tumors like craniopharyngiomas.

7. Use according to any of the preceding claims, wherein the anticancer agent consisting of a radiosensitizing anticancer compound or a mixture of anticancer compounds containing at least one radiosensitizing anticancer compound, said anticancer compound(s) being chosen in the group comprising 5-fluorouracil (5-FU), platinum agents, such as carboplatin and cisplatin, taxanes, such as docetaxel and paclitasel.

8. Use according to claim 7, wherein the anticancer agent is 5-fluorouracil.

9. Use according to claim 1, wherein a neuroprotective compound is added.

10. Use according to claims 1 or 2, wherein the microspheres are suspended in a sterile solution containing between 1 and 1.5%, preferably 1.25% weight/volume, of a viscosity modifier, such as sodium carboxymethyl cellulose, between 0.5 and 1.5%, preferably 1%, of a surfactant, such as Polysorbate 80®, and between 3.5 and 4.5%, preferably 4%, of an isotonicity agent, for example mannitol.

11. Use according to claim 10, wherein the suspension contains 3 ml of the sterile solution and 700 to 800 mg of biodegradable microspheres.

12. Use according to claim 8, wherein the amount of 5-FU is of between 50 and 200 mg.

13. Use according to claim 1, wherein the microspheres are administered by one or more repeated stereotactic injections.

14. Use according to claim 1, wherein the administration of microspheres is followed by a radiotherapy.

15. Use according to claim 1, wherein the microspheres are prepared by a method consisting in preparing an organic phase in which the anticancer agent and the polymer are dispersed in an organic solvent, emulsifying the organic phase and an aqueous phase, extracting the organic solvent by adding water and finally filtering the suspension of microspheres thus obtained.
